(19) United States
(12) Patent Application Publication
(10) Pub. No.: US 2026/0057978 A1
(43) Pub. Date: Feb. 26, 2026

(54) MANAGING A PROCESSING LOCATION OF PATIENT SPECIFIC DATA

(71) Applicant: Siemens Healthineers AG, Forchheim (DE)

(72) Inventors: Bjoern NOLTE, Fuerth (DE); Lutz DOMINICK, Eggolsheim (DE)

(73) Assignee: Siemens Healthineers AG, Forchheim (DE)

(21) Appl. No.: 19/308,771

(22) Filed: Aug. 25, 2025

(30) Foreign Application Priority Data
Aug. 26, 2024 (DE) 10 2024 208 099.3

(51) Int. Cl. G16H 10/60; G16H 30/40

(52) U.S. Cl. CPC G16H 10/60; G16H 30/40

(57) ABSTRACT

The application relates to a system configured to manage processing of patient-specific data. The system includes a pretrained large language model configured to: receive, as an input, processing rules including legal constraints as to which location a processing of the patient-specific data is allowed; process the input to determine a processing location where an application configured to process the patient-specific data can process the patient-specific data meeting the legal constraints; and provide, as output, the processing location for further use.

### CROSS-REFERENCE TO RELATED APPLICATION(S)

The present application claims priority under 35 U.S.C. § 119 to German Patent Application No. 10 2024 208 099.3, filed Aug. 26, 2024, the entire contents of which is incorporated herein by reference.

### FIELD

The present application relates to a system configured to manage a processing of patient-specific data and to the corresponding method for operating the system. Furthermore, a non-transitory computer-readable medium storing a computer program comprising program code is provided.

### BACKGROUND

Agreements and contracts regulate how data is to be handled within a clinic. The clinic can regulate if equipment and algorithm vendors can run applications only locally within the computer network inside the hospital(s) or clinic(s). There are cases where permission is granted for a private virtual network and there are cases where certain applications are allowed to run in a vendor-controlled cloud infrastructure potentially from a large cloud service provider. The applications process patient-specific data such as medical images of the patients generated with imaging systems such as Magnetic Resonance Imaging (MRI) systems, or CT (Computer Tomography). The patient-specific data could also include physiological data of the patient.

The complicated contractual obligations for the processing of patient specific data are today manually reviewed and a statical configuration is performed on where applications are run, where data is held and processed and where results, such as updated ML (Machine Learning) models, client Apps presenting their results are located. The ML models can be used in the applications for image processing or other data analysis steps. Software running the applications cannot be used “as is” any longer as especially a location of the data processing with the software provided in the application can depend on different factors such as legal constraints provided by the country in which the patient-specific data were generated, contracts between the vendor of the software and the clinic. As a consequence, a planning of the processing of the patient-specific data can become difficult as many different processing rules concerning the processing of the patient-specific data have to be considered.

### SUMMARY

Accordingly, a need exists to overcome the above identified problems and to allow a processing of patient-specific data in an effective way such that any legal constraints are met.

This need is met by the features of the independent claims. Further aspects are described in the dependent claims.

According to a first aspect a system is provided configured to manage a processing of patient-specific data, the system comprising a pretrained large language model configured to receive as an input processing rules including legal constraints at which location a processing of the patient-specific data is allowed. The language model is configured to process the input in order to determine a processing location where an application configured to process the patient-specific data can process the patient-specific data meeting the legal constraints and the language model is further configured to provide as an output the determined processing location for further use.

A large language model can parse the legal constraints provided in the processing rules and can produce an appropriate configuration where the patient-specific data should be processed by the application. Accordingly, the system can determine the processing configuration including the location where the processing of the patient-specific data should be carried out.

Furthermore, the corresponding method is provided which operates as discussed above. In addition, a computer program comprising program code is provided to be executed by at least one processing unit of a system wherein execution of the program code causes the at least one processing unit to carry out a method as mentioned above or as discussed in detail below.

It is to be understood that the features mentioned above and features yet to be explained below can be used not only in the respective combinations indicated, but also in other combinations or in isolation without departing from the scope of the present invention. Features of the above-mentioned aspects and embodiments described below may be combined with each other in other embodiments unless explicitly mentioned otherwise.

### BRIEF DESCRIPTION OF THE DRAWINGS

The foregoing and additional features and effects of the application will become apparent from the following detailed description when read in conjunction with the accompanying drawings in which like reference numerals refer to like elements.

FIG. 1 shows a schematic architectural view of a system which is configured to determine a processing configuration meeting legal constraints.

FIG. 2 is a schematic view of the system of FIG. 1 where a movement of an application including an artificial neural network to the premises becomes necessary due to restrictions provided in the processing rules.

FIG. 3 shows a schematic view of an example flowchart of a method carried out by a system shown in FIG. 1.

FIG. 4 shows a schematic view of a system configured to determine the processing location of the patient-specific data.

FIG. 5 shows a further schematic view of a system configured to automatically deploy an application using patient-specific data at a location meeting processing rules including legal constraints for the processing.

### DETAILED DESCRIPTION

In the following, embodiments of the present invention will be described in detail with reference to the accompanying drawings. It is to be understood that the following description of embodiments is not to be taken in a limiting sense. The scope of the present invention is not intended to be limited by the embodiments described hereinafter or by the drawings, which are to be illustrative only.

The drawings are to be regarded as being schematic representations, and elements illustrated in the drawings are not necessarily shown to scale. Rather, the various elements are represented such that their function and general purpose becomes apparent to a person skilled in the art. Any connection or coupling between functional blocks, devices, components of physical or functional units shown in the drawings and described hereinafter may also be implemented by an indirect connection or coupling. A coupling between components may be established over a wired or wireless connection. Functional blocks may be implemented in hardware, software, firmware, or a combination thereof. Independent of the grammatical term usage, individuals with male, female, or other gender identities are included within the term.

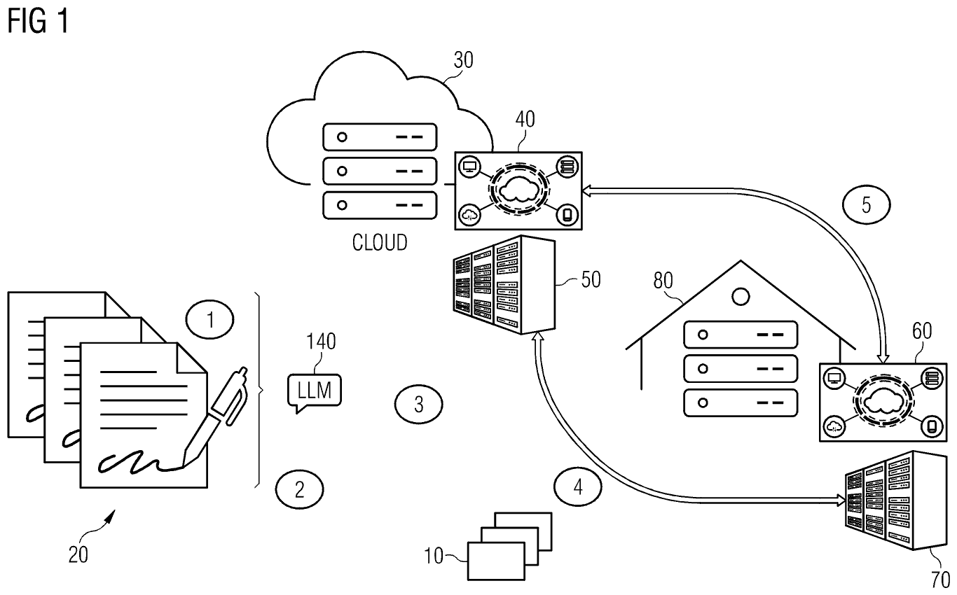

FIG. 1 shows a schematic view of a system in which a specially trained neural network, a large language model 140 is configured to determine a processing location for patient-specific data 10 such as medical images obtained from a patient using imaging systems such as MRI, CT, PET or ultrasound. Furthermore, the patient-specific data can include physiological data obtained from a blood sample of the patient. The large language model 140 is a specially pretrained model which has been trained using processing rules 20 which include the legal constraints available for the processing of the patient-specific data. The processing rules 20 can include the appropriate contracts, data usage permissions, mandate data potentially per patient and general customer data provided for the corresponding hospital or clinic. This data is fed as background to a general LLM as also shown by step 1. The processing rules can furthermore include application policies provided by an application such as application 40 provided in a cloud environment 30 or application 60 provided at the premises 80 which is used to process the patient-specific data 10. Furthermore, country or region-specific rules can be provided in the processing rules 20 which indicate whether and where the processing of personal data such as patient data is allowed or not. Accordingly, in addition to a general training of the LLM 140 a case-specific training such as a supervised learning is carried out using known processing rules fed to the system. The collection of any legal constraints contained in the processing rules are parsed by the LLM 140 in step 2. The LLM 140 can include Generative pretrained transformers such das Chat GPT, LIAMA, LAMBDA; BERT, Claude, Cohere, Ernie, Falcon 40B, GPT-4, Orca, Palm, StableLM.

The trained LLM 140 is then instructed to use the collection of the processing rules to produce the appropriate configuration instructions to run the clinical applications 40 or 60 either in the cloud 30 or within the premises 80 of the clinic where the patient-specific data were generated, accordingly an output is generated including the determined processing location (step 3). The cloud environment 30 and the premises each have the hardware or processing capacity 50, 70 such as memory to store and run the applications 40, 60. The applications 40, 60 may be identical, however it is also possible that the application used to process the patient-specific data provided in the cloud 30 can differ from the corresponding application 60 provided at the premises 80. The application 40, 60 can be implemented as software as a service, SAAS or could be a native software application which is run in the appropriate environment according to the narrative from the constraints provided in the processing rules 20. The environment and the processing rules may change over time, especially when specific circumstances appear. These circumstances can include patient transfer, patient change of consent, policy changes of the clinic, policy changes in laws and regulations or policy changes from the vendor which provides the application for the processing.

The LLM 140 then produces an appropriate output to configure the applications 40 or 60 to be instantiated in the right environment and to host the data in the right environment so that the patient-specific data are either processed in the cloud 30 or at the premises 80.

The application and the corresponding processing capabilities including memory can be provided as a container-based service where a container orchestration system such as Kubernetes is used for software deployment, scaling, and management. Accordingly, the application 40 or 60 and the hardware 50 and 70 could be provided as a container and the container could be deployed in the cloud environment or at the premises. The container could be provided at each of the locations shown in FIG. 1, the cloud 30 and the premises 80, or could be moved from one location to the other based on the output given by the LLM 140. If data appears such as new patients or patients with other permission parameters and new data sets of patient-specific data are produced, the LLM 140 then uses this additional information to decide if the applications need to be shifted from one to another environment such as from the private cloud infrastructure to on premise or vice versa. Furthermore, the country specific legal constraints may change, or new patient data or patients with other permissions may be present so that it might become necessary to shift the application or SaaS from the cloud infrastructure to the premises as indicated by steps 4 or 5 in FIG. 1.

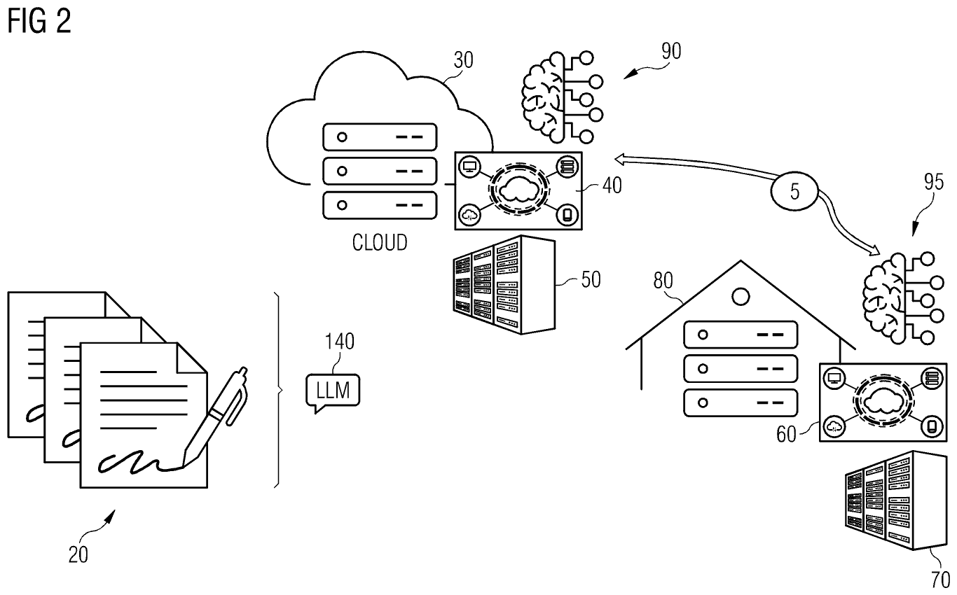

FIG. 2 shows a similar situation as shown in FIG. 1 where the application uses a machine learning module such as a neural network 90 to which the patient-specific data is applied. By way of example the neural network 90 can be configured to identify malignant parts in medical images. The processing rules can include constraints as far as the use of medical images or patient-specific data for machine learning is allowed or not. In this scenario the neural network 90 generates a data model using an appropriate machine learning algorithm wherein the data model may be initially hosted in the cloud environment 30. As constraints are fed and analyzed by the LLM 140, it may happen that restrictions are detected which prohibits the further training of the neural network 90 in the cloud environment. Accordingly, the LLM 140 can generate instructions for the configuration of the application training environment where the latest and best model of the neural network, the neural network 90 is moved to the premises 80 for deployment (step 5). The neural network 95 may then be further trained locally within premises 80. Neural network 95 can correspond to network 90, but it can also be an updated version which has been newly trained locally. If no further data sets for training are available at the premises, the latest data model can be analyzed to contain no private or patient-specific data and the neural network 95 may be transferred back to the cloud environment 30. This step can be done with LLM technology or other appropriate machine learning technologies. Furthermore, it is possible that if there is private patient data still persistent in the data model used in neural network 95 or 90, an exception may be raised and logged.

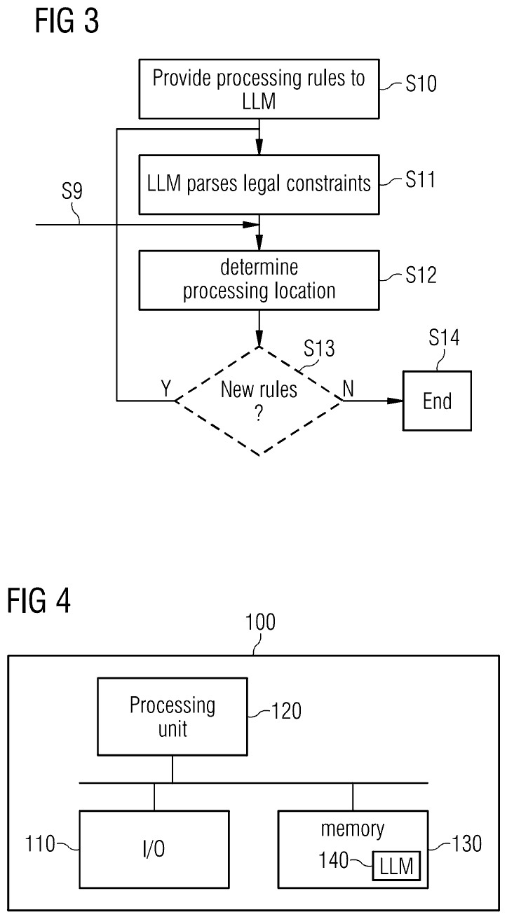

FIG. 3 shows some of the steps carried out in a system as shown in FIGS. 1 and 2. In step S10 the processing rules including the legal constraints are provided to the language model LLM which was pretrained in a supervised training using known processing rules. In step S11 the LLM parses the legal constraints. When an instruction is received including patient specific data and possibly all the processing rules, if not already present in the LLM (arrow S9), the knowledge of the processing rules is used to determine a processing location in S12. This can include the step of configuring the application to be instantiated in the right environment, at the cloud 30 or within premises 80. Furthermore, in a further optional step it can be determined in step S13 whether new processing rules or new patient data with other permission parameters are received. If this is the case the method returns to step S11 where the LLM parses the legal constraints in order to be able to determine a processing location meeting any legal constraints. If no new rules are detected the method ends in step S14.

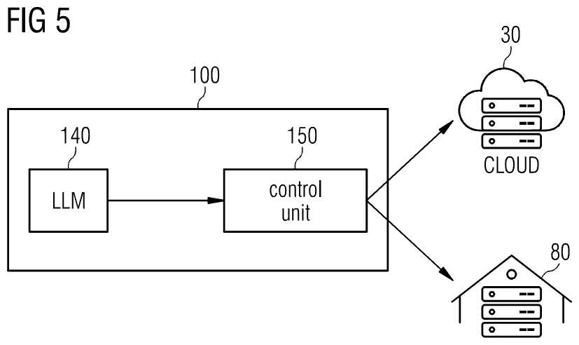

FIG. 4 shows a schematic view of a system 100 configured to manage the processing of the patient-specific data and which can provide as output a determined processing location as discussed above. The system 100 can include an interface 110 provided for receiving the processing rules and provided for outputting the determined processing location and providing the processing rules to the LLM 140. The system furthermore comprises a processing unit 120 responsible for the operation of the system 100. The processing unit 120 can comprise one or more processors and can carry out instructions stored on a memory 130 wherein the memory also includes the pre-trained LLM 140.

In general, the system can be configured to simply output the determined processing location or application environment where the patient-specific data and the application should be located during processing. This output can then be used by a user of the system to select the determined processing environment for the patient specific data.

FIG. 5 shows a situation where the system 100 furthermore includes a control unit 150 which receives the output of the LLM and which directly uses this output as instructions to configure the processing in such a way that the legal constraints are met. The application, the processing capacities and the software can be implemented as a container-based service wherein a container is used to provide the processing of the patient-specific data. The control unit can be part of a container manager or can instruct a container manager to carry out the processing in the cloud environment 30 or at the premises 80, and can instantiate the container at the determined location if needed.

From the above explanation some general conclusions can be drawn:

The system can be configured to provide the determined processing location for further use. Furthermore, it can mean that a user uses the output from the LLM 140 to trigger the processing of the patient-specific data. It can also mean that the system includes a control unit which receives as input the output of the determined processing location and triggers a processing of the patient-specific data at the determined location. Here a complete automated system is provided where no user input is required.

The control unit may be configured to move the patient-specific data and the application needed for the processing to the determined processing location if the corresponding data or application is not available at the determined processing location.

The application can be provided as a container-based service and the control unit can be configured to initiate a movement of the container providing the processing of the patient-specific data as container-based service to the determined processing location.

The pretrained large language model can be configured to determine, based on the legal constraints, as processing location either the premises where the patient-specific data were generated, such as the clinic or hospital or may determine a cloud-based processing environment outside the premises for the processing.

The processing rules can include country or region-specific rules where the processing of the patient-specific data is allowed or not. Furthermore, the processing rules could include legal constraints set up by the premises where the patient-specific data is generated, it could include legal constraints set up by a manufacturer providing an apparatus with which the patient-specific data is generated, and/or the patient-specific rules which are set up between the patient and the premises or hospital where the patient-specific data is generated.

The LLM can provide the determined processing location as part of a complete processing configuration which is used to process the patient-specific data by the application.

The patient-specific data may include image data of the patient obtained by an imaging system or may include physiological data of the patient.

The control unit can be configured to detect a change in the input processing rules over time and if a changed input processing rule is detected it is input into the pretrained large language model which processes the changed input processing rule and provides as output an adapted processing location based on the changed input processing rule.

The application may include a trained neural network applied to the patient-specific data wherein the control unit can be configured to move a latest version of the trained neural network to the premises where the patient-specific data has been generated when the processing rules indicate that a cloud-based training of the trained neural network with the patient-specific data is not allowed.

Summarizing an improved method for determining a processing location of patient-specific data is provided.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RA4), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A system configured to manage a processing of patient-specific data, the system comprising:
a pretrained large language model configured to
receive processing rules as an input, the processing rules including legal constraints as to which location a processing of the patient-specific data is allowed,
process the input to determine a processing location at which an application configured to process the patient-specific data is able to process the patient-specific data meeting the legal constraints, and
provide, as output, the processing location for further use.

2. The system of claim 1, further comprising:
a control unit configured to
receive the output of the pretrained large language model, and
trigger processing of the patient-specific data at the processing location.

3. The system of claim 2, wherein the control unit is configured to move the patient-specific data and the application to the processing location.

4. The system of claim 2, wherein
the application is provided as a container based service, and
the control unit is configured to initiate movement of a container providing the processing of the patient-specific data as the container based service, to the processing location.

5. The system of claim 1, wherein the pretrained large language model is configured to determine, based on the legal constraints, the processing location as either a premises where the patient-specific data was generated or a cloud based processing environment outside the premises.

6. The system of claim 1, wherein the processing rules include at least one of:
country or region specific rules where the processing of patient-specific data is allowed,
legal constraints set up by a premises where the patient-specific data is generated,
legal constraints set up by a manufacturer providing an apparatus with which the patient-specific data is generated, or
patient-specific rules set up between a patient and the premises where the patient-specific data is generated.

7. The system of claim 1, wherein the pretrained large language model is configured to provide the processing location as part of a processing configuration used to process the patient-specific data by the application.

8. The system of claim 1, wherein the patient-specific data includes image data of a patient obtained by an imaging system.

9. The system of claim 1, wherein the pretrained large language model has been trained with a general training and a case specific training in which input processing rules including legal constraints for a location at which a processing of the patient-specific data is allowed were used in a supervised learning.

10. The system of claim 2, wherein
the control unit is configured to
detect a change in the processing rules over time, and
in response to detecting a changed input processing rule, input the changed input processing rule into the pretrained large language model, which is configured to process the changed input processing rule and to provide, as output, an adapted processing location based on the changed input processing rule.

11. The system of claim 3, wherein
the application includes a trained neural network applied to the patient-specific data, and
the control unit is configured to move a latest version of the trained neural network to a premises where the patient-specific data has been generated, when the processing rules indicate that a cloud based training of the trained neural network with the patient-specific data is not allowed.

12. A method for managing a processing of patient-specific data, the method comprising:
inputting, as an input, processing rules into a pretrained large language model of a system, the processing rules including legal constraints as to which location that processing of the patient-specific data is allowed;
processing, by the pretrained large language model, the input to determine a processing location where an application configured to process the patient-specific data is able to process the patient-specific data meeting the legal constraints; and
outputting the processing location for further use.

13. The method of claim 12, further comprising:
inputting the processing location into a control unit; and
triggering, by the control unit, the processing of the patient-specific data at the processing location.

14. The method of claim 13, further comprising:
moving, by the control unit, the patient-specific data and the application to the processing location.

15. The method of claim 13, wherein
the application is provided as a container based service, and
the method further includes initiating, by the control unit, movement of a container providing the processing of the patient-specific data as the container based service to the processing location.

16. The method of claim 12, wherein the pretrained large language model determines, as the processing location, either a premises where the patient-specific data was generated or a cloud based processing environment outside the premises.

17. The method of claim 12, wherein the pretrained large language model provides the processing location as part of a processing configuration used to process the patient-specific data by the application.

18. The method of claim 12, wherein the pretrained large language model has been trained with a general training and a case specific training in which input processing rules including legal constraints for a location at which a processing of the patient-specific data is allowed were used in a supervised learning.

19. The method of claim 13, further comprising:
detecting, by the control unit, a change in the processing rules over time;
inputting, in response to detecting a changed input processing rule, the changed input processing rule into the pretrained large language model which processes the changed input processing rule; and
providing, as output, an adapted processing location based on the changed input processing rule.

20. The method of claim 12, wherein
the application includes a trained neural network applied to the patient-specific data, and
the method further includes moving, by a control unit, a latest version of the trained neural network to a premises where the patient-specific data has been generated, when the processing rules indicate that a cloud based training of the trained neural network with the patient-specific data is not allowed.

21. A non-transitory computer-readable medium storing a computer program including program code that, when executed by at least one processing unit of a system, causes the at least one processing unit to carry out the method of claim 12.
